(19) United States
(12) Patent Application Publication
(10) Pub. No.: US 2018/0061049 A1
(43) Pub. Date: Mar. 1, 2018

(54) SYSTEMS AND METHODS FOR ANALYZING IN VIVO TISSUE VOLUMES USING MEDICAL IMAGING DATA

(71) Applicant: MAYO FOUNDATION FOR MEDICAL EDUCATION AND RESEARCH, ROCHESTER, MN (US)

(72) Inventors: Richard A. Robb, Rochester, MN (US); Srinivasan Rajagopalan, Rochester, MN (US); Ronald A. Karwoski, Rochester, MN (US); Brian J. Bartholmai, Rochester, MN (US)

(21) Appl. No.: 15/803,230

(22) Filed: Nov. 3, 2017

Related U.S. Application Data
(63) Continuation of application No. 14/115,688, filed on Feb. 5, 2014, now abandoned.
(60) Provisional application No. 61/518,424, filed on May 5, 2011.
(60) Provisional application No. 61/483,881, filed on May 9, 2011.

(51) Int. Cl. G06T 7/00; A61B 6/03; A61B 5/055; A61B 5/00; G06T 11/20; A61B 6/00

(52) U.S. Cl. CPC G06T 7/0012; A61B 6/037; G06T 7/11; A61B 5/055; A61B 5/7264; G06T 11/206; G06T 2207/30101; A61B 6/5205; G06T 7/62; A61B 6/032; A61B 5/7267; G06T 2207/10072; G06T 2207/30061; A61B 6/5217

(57) ABSTRACT

Computer-aided methods and computer-based systems designed to elicit information from imaging data of a volume of in vivo tissue to facilitate clinical determinations and/or pathological evaluation.

### CROSS-REFERENCE TO RELATED APPLICATIONS

This application is a continuation and is based on, claims the priority to, and incorporates herein by reference U.S. patent application Ser. No. 14/115,688, entitled “SYSTEMS AND METHODS FOR ANALYZING IN VIVO TISSUE VOLUMES USING MEDICAL IMAGING DATA,” filed Feb. 5, 2014, PCT Application Serial No. PCT/US2012/036802, entitled “SYSTEMS AND METHODS FOR ANALYZING IN VIVO TISSUE VOLUMES USING MEDICAL IMAGING DATA,” filed May 7, 2012, U.S. Provisional Patent Application Ser. No. 61/518,424, entitled “SYSTEMS AND METHODS FOR ANALYZING IN VIVO TISSUE VOLUMES USING MEDICAL IMAGING DATA,” filed May 5, 2011, and U.S. Provisional Patent Application Ser. No. 61/483,881, entitled “SYSTEMS AND METHODS FOR ANALYZING IN VIVO TISSUE VOLUMES USING MEDICAL IMAGING DATA,” filed May 9, 2011.

### STATEMENT REGARDING FEDERALLY SPONSORED RESEARCH

Not applicable.

### BACKGROUND OF THE INVENTION

The present application is directed to systems and methods for analyzing in vivo tissue volumes using medical imaging data.

Medical imaging has become a mainstay of modern clinical research and medicine. Medical images provide can provide a researcher or clinician with a wealth of information about in vivo anatomical structure and physiological performance and, thereby, provide key clinical indicators and diagnostic parameters. In fact, one substantial challenge to the effective use of the wide and varied information available through non-invasive imaging is the ability to analyze, parse, and ultimate use particular pieces of the vast information provided in a given medical image to drive clinical decisions. Recognizing this challenge, substantial efforts have been made to build systems and methods that attempt to facilitate the analysis of medical imaging data and assist the clinician or researcher in using the information contained in the medical imaging data.

For example, one category of tool developed to aid the radiologist in image analysis is generally referred to as a computer-aided diagnosis (CAD) systems.

CAD systems have been developed that attempt to analyze images, for example, images generated during a mammographic screening, and provide feedback to the radiologist and/or other physician indicating potential markers of malignancy that should be reviewed. Over the years, these systems have been built, rebuilt, and refined, such that many now include complex neural networks and various analysis algorithms with which to analyze the images.

While these CAD systems are a useful tool for aiding a radiologist and/or other physician with reviewing the images acquired during screening processes, proper diagnosis by the radiologist and/or other physicians requires consideration of all available information, such as personal and familial medical histories, and use of this information as a lens through which to review the images and the CAD indicators. Due to the fact that this synthesis of information and ultimate analysis procedure is reliant upon the radiologist and/or other physicians, even when aided with CAD systems, the efficacy of image screening is highly dependent upon the subjective abilities of radiologists and/or other physicians to synthesize and analyze information.

Similarly, an oft-cited survey paper on the “Computer Analysis of Computed Tomography Scans of the Lung” (IEEE TMI 25(4), April 2006: 385-405, states “First step toward more advanced processing schemes have been taken, but in the computer analysis of Diffuse Pulmonary Lung Disease, the question on what exactly to aim for and how to achieve it is still open.” The paper continues, “Classification and quantification of interstitial lung disease is difficult, and even experienced chest radiologists frequently struggle with different diagnoses.” However, “Automated schemes that indicate a percentage of affected lung or the probability of a certain disease would certainly be welcome, but require more research.” This portion of the paper concludes, “A quick analysis of the roughly 300 publications considered for this survey reveals that the amount of publications in this field has grown by a factor 1.5 per year over the past five years.” However, despite the proliferation of academic hype on the strategies for quantifying diseases such as lung diseases using medical images, none of the currently-available systems or methods is readily capable of meeting the wide and variable clinical challenges.

As a further example, a joint recommendation of the American Thoracic Society and European Respiratory Society (ATS/ERS) specifies standardized definition and criteria for the diagnosis of diffuse pulmonary lung diseases (DPLD). The recommendation stresses the importance of collaborative clinico-radiologic-pathologic diagnosis whereby a patient's lung wellness is assessed through multidisciplinary iterative discussions among clinicians, radiologists and pathologists. This multidisciplinary diagnosis has been reinforced by other thoracic societies and a number of pilot studies have confirmed the efficacy of such interactions in diagnosing lung disease/wellness.

A study described in Flaherty K R, King T E, Raghu G, et al. Idiopathic Interstitial Pneumonia What is the effect of a multidisciplinary approach to diagnosis? Am J Respir Crit Care Med, 2004; 170: 904-910 reveals the DPLD diagnostic disparity between physicians based in academic and community centers. Such disturbing disparity could (at times, irreversibly) compromise patient care, and the optimal assessment of disease through the use of multi-site, multidisciplinary subspecialty assessment is prohibitively expensive and practically unfeasible.

Despite its efficacy, the consensus-based diagnosis has not attained clinical familiarity, let alone integration into routine practice. The study of Wells A U, Hogaboam C M. Update in diffuse parenchymal lung disease 2007, Am J Respir Crit Care Med, 2008; 177: 580-584 shows that 28 percent of pulmonologists who responded to a survey on A TS/ERS recommendation were not aware of its existence. Beyond the traditional barriers of physician adherence to clinical practice guidelines, the ATS/ERS recommendation lacks practicality. It is impractical in typical clinical, or even in multispecialty academic settings, to routinely establish consensus via group discussion among multiple physicians. Even if this was possible, the differences in experience, knowledge and potential unblinded bias could adversely affect the accuracy and consistency of such a consensus diagnosis.

Therefore, it would be desirable to provide systems and methods to aid in the analysis of in vivo tissue volumes using medical imaging data. Furthermore, it would be desirable to have systems and methods that facilitate diagnostic consistency. Further still, it would be desirable to have systems and methods that enable the detection of clinically relevant indicators across multiple images or a historical record or time-course of images.

### SUMMARY OF THE INVENTION

The present invention overcomes the aforementioned drawbacks by providing a computer-aided methods and computer-based systems designed to elicit information from imaging data of a volume of in vivo tissue to facilitate clinical determinations and/or pathological evaluation.

In one aspect, the present invention provides a computer-readable medium having encoded thereon instructions which, when executed by at least one processor, execute a method for displaying medical imaging data including the steps of receiving medical image data including intensity-based tissue texture appearance data having a plurality of data types each representative of a different tissue type. The method conducts segmentation to delineate the different tissue types and determines a plurality of tissue groups by classifying the data types and differentiating the tissue types using a similarity metric. The intensity-based tissue texture appearance data are clustered in the tissue groups using an unsupervised clustering technique, and the amount of data in each tissue group is determined. The method generates a report including a plurality of shapes concurrently, the area of each shape being proportional to the amount of data in a different one of the tissue groups.

In another aspect, the present invention provides a computer-readable medium having encoded thereon instructions which, when executed by at least one processor, execute a method for displaying medical imaging data including the steps of receiving medical image data including tissue data representative of a plurality of regions of interest each having a volume. The tissue data has a plurality of data types each representative of a different tissue type. The method conducts segmentation to delineate the different tissue types and determines a plurality of tissue groups by classifying the data types and the different tissue types. The tissue data are clustered in the tissue groups, and the amount of the tissue data in each tissue group is determined. The method generates a report including a circular-shaped glyph including a plurality of circular sectors, and each circular sector has an overall area proportional to the volume of a corresponding one of the regions of interest. Each circular sector includes a plurality of radially offset arcuate segments together defining the overall area of the circular sector, and each radially offset arcuate segment has an area proportional to the amount of tissue data in a different one of the tissue groups within the corresponding one of the regions of interest.

These and other features and advantages of the present invention will become apparent upon reading the following detailed description when taken in conjunction with the drawings.

### BRIEF DESCRIPTION OF THE DRAWINGS

FIG. 1 is a schematic diagram of a system in accordance with the present invention;

FIG. 2 is a visualization in accordance with the present invention;

FIGS. 3A and 3B are a series of visualizations in accordance with the present invention;

FIG. 4A is a further visualization in accordance with the present invention;

FIG. 4B illustrates correlations of visualizations with anatomical images in accordance with the present invention;

FIGS. 5 and 6 are further series of visualizations in accordance with the present invention;

FIG. 7 is a set of further visualizations, including maximum disease projections for a number of independent patient-specific datasets in accordance with the present invention;

FIG. 8 is a flow chart illustrating processes of an exemplary algorithmic subsystem of a data analysis and visualization system in accordance with the present invention;

FIG. 9 is a series of graphs illustrating correlations that can be visualized in accordance with the present invention;

FIG. 10 is a graph showing mean intra-cluster and inter-exemplary Cramer Von Mises (CVM) values for the computed class;

FIG. 11 is a series of images illustrating representative results of a CVM-based lung tissue classification;

FIG. 12 is a representative visualization summarizing the holistic distribution of a patterns across the lung lobes; and

FIG. 13 is another series of representative visualizations illustrating the visualization's capability to readily convey information across a series of medical image data sets.

### DETAILED DESCRIPTION OF THE INVENTION

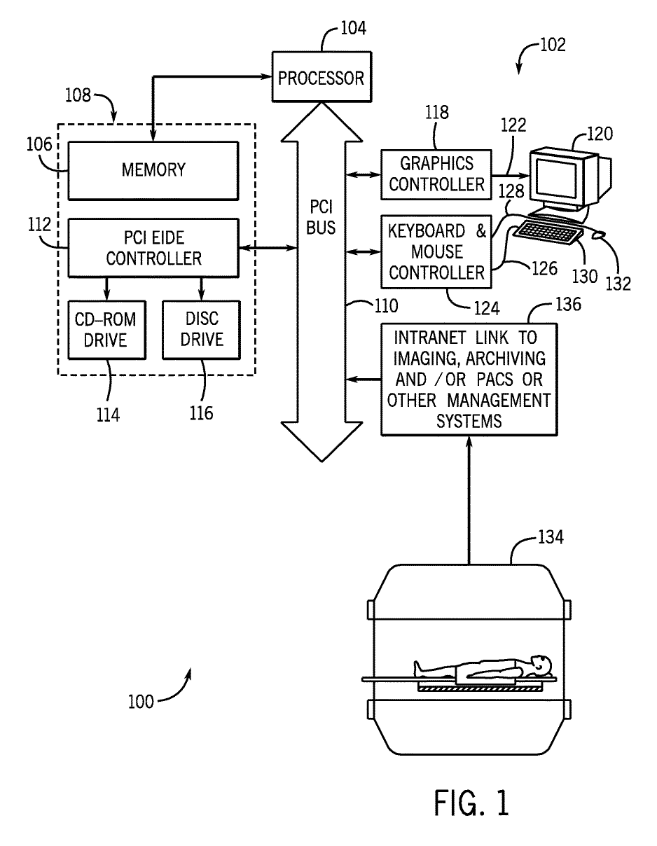

Referring now to FIG. 1, an analysis and imaging system 100 for conducting analysis in accordance with the present invention is illustrated. The system includes computer workstation 102 includes a processor 104 that executes program instructions stored in a memory 106 that forms part of a storage system 108. The processor 104 is a commercially available device designed to operate with available operating systems. It includes internal memory and I/O control to facilitate system integration and integral memory management circuitry for handling all external memory 106. The processor 104 also has access to a PCI bus driver that facilitates interfacing with a PCI bus 110.

The PCI bus 110 is an industry standard bus that transfers data between the processor 104 and a number of peripheral controller cards. These include a PCI EIDE controller 112 which provides a high-speed transfer of data to and from an optical drive 114 and a disc drive 116. A graphics controller 118 couples the PCI bus 110 to a display 120 through a standard display connection 122, and a keyboard and a mouse controller 124 receives data through respective connections 126, 128 that is manually input through a keyboard 130 and mouse 132. For example, the display 120 may be a monitor, which presents an image measurement graphical user interface (GUI) that allows a user to view imaging results and may also act as an interface to control an imaging system 134. Specifically, the PCI bus 110 may also serve connect to a the imaging system 134 directly or may receive medical imaging data through an intranet 136 that links workstations, a department picture archiving and communication system (PACS), or an institution image management system.

As will be described, the imaging system 134 may include any of a wide variety of medical imaging systems, such as magnetic resonance imaging (MRI) systems, computed tomography (CT) systems, positron emission tomography (PET) systems, single photon emission computed tomography (SPECT) systems, and many other systems. That is, the present invention is not specifically limited to or for use with one particular imaging modality or image data type. Rather, as will be explained, the present invention is useful with a wide variety of imaging modalities and data types capable of eliciting information pertaining to volumes within a subject. In particular, the present invention provides systems and methods that provide a holistic, iconic, view-independent summary of an extent of a spatiotemporal distribution of the normal and abnormal tissues as abstracted from the analysis of multi-dimensional volumetric representations of a patient-specific tissue volume, such as the lung.

As will be described, using such information, the present invention provides a computational framework that learns the decision rules of the multiple specialties, embraces evidence-based clinical practice guidelines, emulates the multidisciplinary consensus process, and provides an integrated, holistic view of the patient's health. To facilitate the following discussion, this general system and method will be referred to as computer aided life informatics for pathology evaluation and rating (CALIPER).

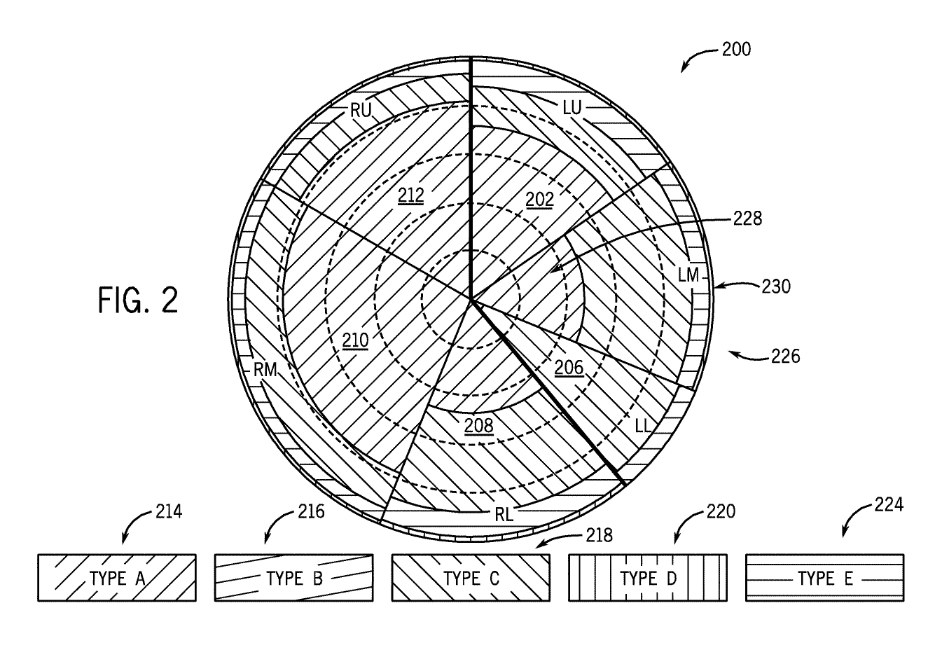

CALIPER has a variety of facets and can be advantageously considered from a variety of different points of view. However, referring to FIG. 2, one facet of CALIPER is the ability to illustrate medical imaging data as a visualization or visual scheme in which an icon-like structure. Such an icon-like structure is partitioned into two not-necessarily equal portions representing the particular spatial sections of the region of interest ROI from which the medical imaging data was acquired, for example the left and right lungs. This icon-like structure, referred to hereafter as a circular-shaped “glyph” 200, can be used to represent portions of the ROI as a set of individual partitions or circular sectors, such as “left upper” (LU) 202, “left middle” (LM) 204, “left lower” (LL) 206, “right lower” (RL) 208, “right middle” (RM) 210, and “right upper” 212. Together, these partitions or circular sectors 202-212 to provide a holistic, iconic, view-independent summary of the extent of regional and temporal distribution of the normal and abnormal tissues in the ROI as abstracted from the analysis of multi-dimensional volumetric representations of a patient-specific tissue volume, in this example, the lung. Specifically, color codes 214, 216, 218, 220, 222 (shown in the figures as cross-hatching codes) are provided to immediately convey spatial and quantification information within the glyph 200. In addition, the glyph 200 is divided in concentric rings to reflect the distributions along the whole lung 226, core 228 and rind 230 of the lung.

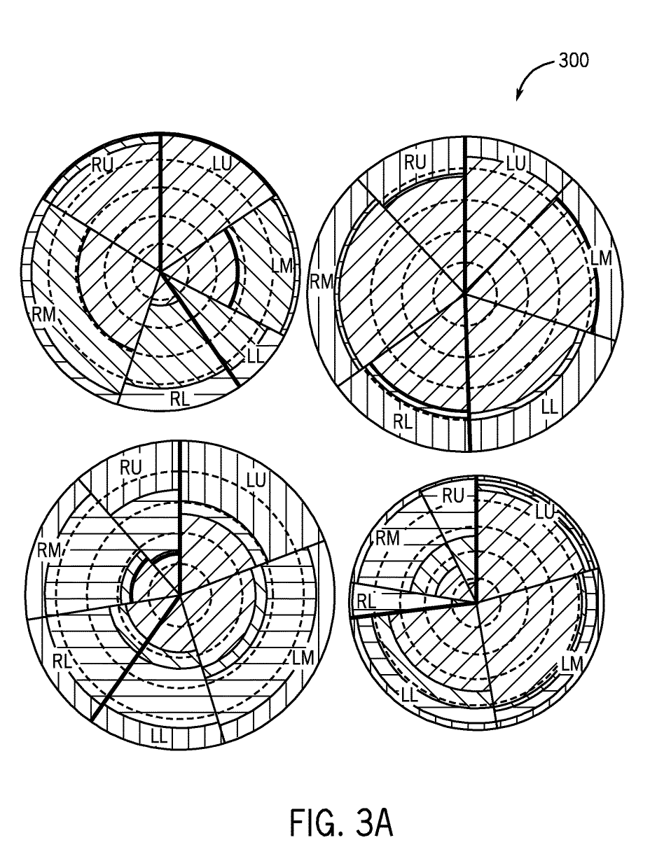

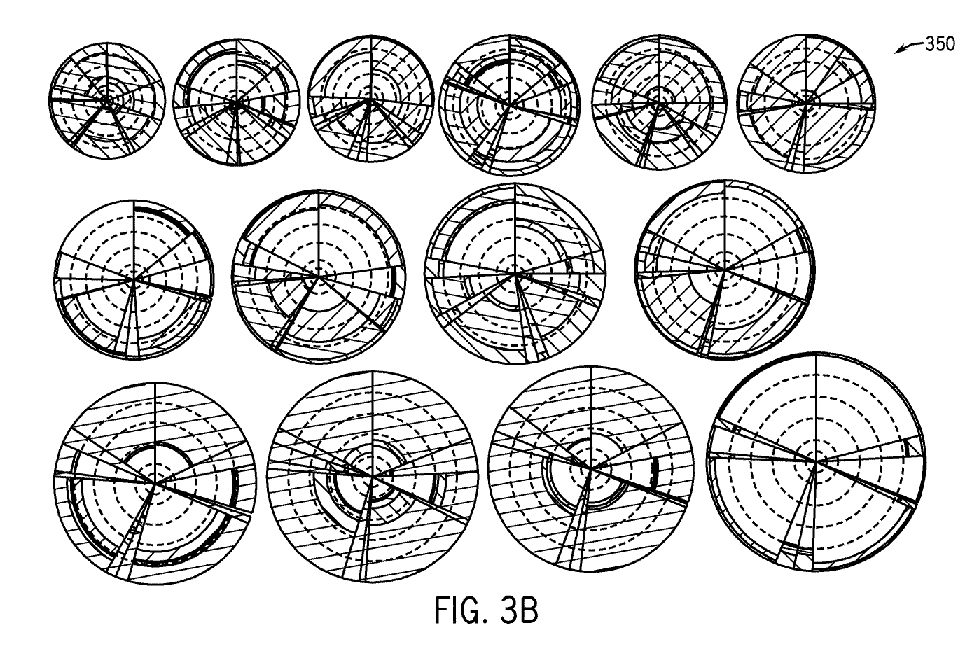

Referring now to FIGS. 3A and 3B, a series of glyphs 300 and 350 may be provided to convey information across a plurality of volumes or a series of images. Specifically, as illustrated in FIG. 3A, the combined partitions are represented in a scale proportional to capture the physiological quantities such as total lung capacity.

On the other hand, FIG. 3B provides a montage showing glyphs from multiple patients. The individual glyphs are scaled proportionately to reflect the underlying physiological quantity such as total lung capacity. The color coding of radially offset arcuate segments defining each of the individual partitions is reflects the distribution of spectrum of normal and abnormal tissue types such that the occupancy of the color codes is proportional to their extent in the underlying lung within that specified hierarchy.

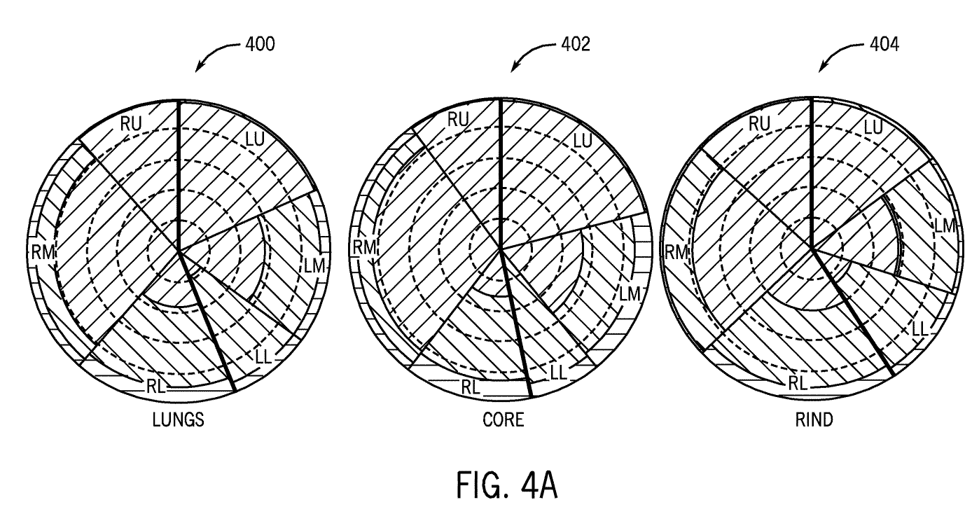

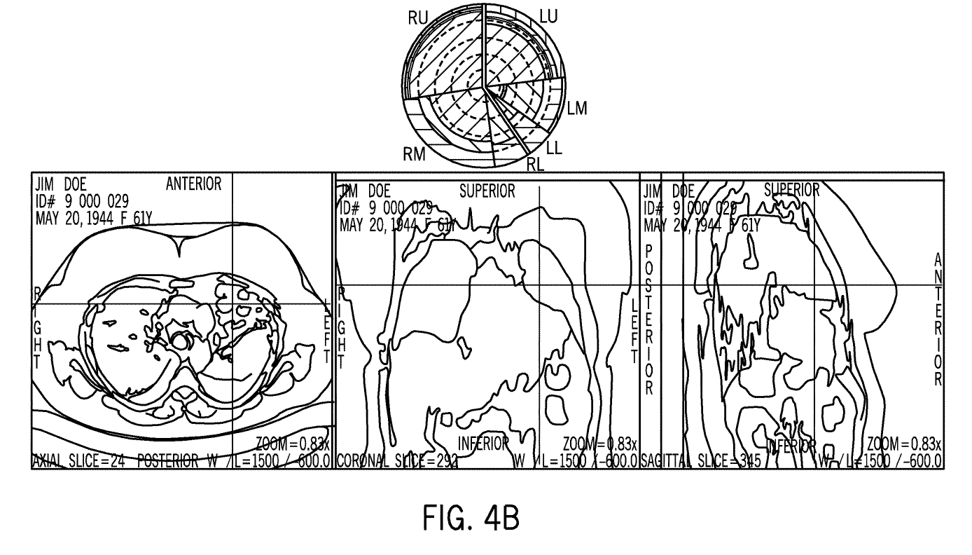

As described above with respect to FIG. 2, the glyph 200 can be divided in concentric rings to reflect the distributions along the whole lung 226, core 228, and rind 230 of the lung. Additionally or alternatively, referring to FIG. 4A, a glyph scheme is illustrated where the glyph with all the above mentioned characteristics are presented to separately illustrate the distributions along the whole lung 400, core 402, and rind 404 of the lung.

Referring now to FIG. 4B, a glyph scheme is illustrated where the respective color coded regions within the different hierarchies on both the left and right partitions are tagged with positional information such that clicking/selecting on that color coding will present the orthogonal positions in the volumetric scan such that best represents the distribution of the selected tissue types. In this regard, a form of global positioning system (GPS) tagging can be performed on the glyphs such that selecting a color coded sector in the glyphs maps the orthogonal sections most representative of the underlying disease state. The cursor in the glyphs indicate the region selected.

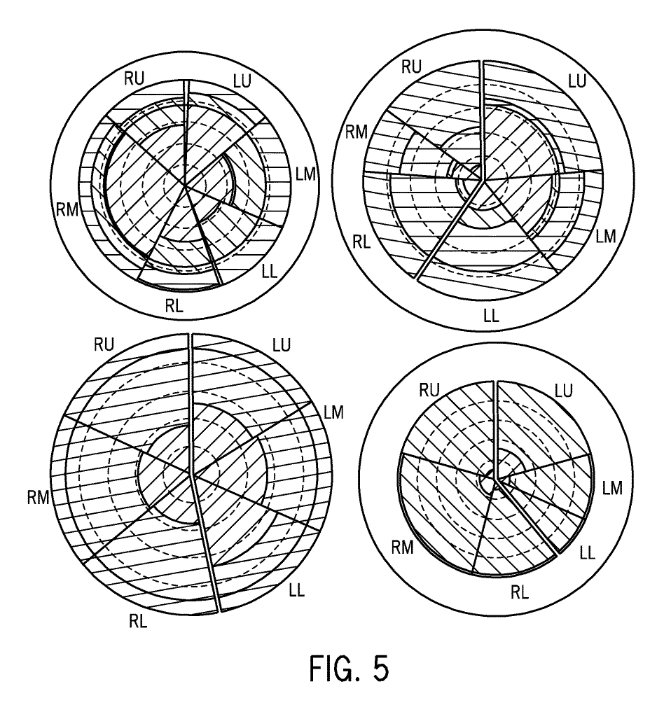

Referring now to FIG. 5, a glyph scheme is provided where the glyph is superimposed with a concentric glyph that represents the predicted lung state of the patient-specified population. The montage shows the glyphs from four different patients each having personalized distribution of circular sectors, arcuate segments, and diseases thereof overlaid with a white ring indicative of the total lung capacity of the population stratified to their age, gender, race, and height.

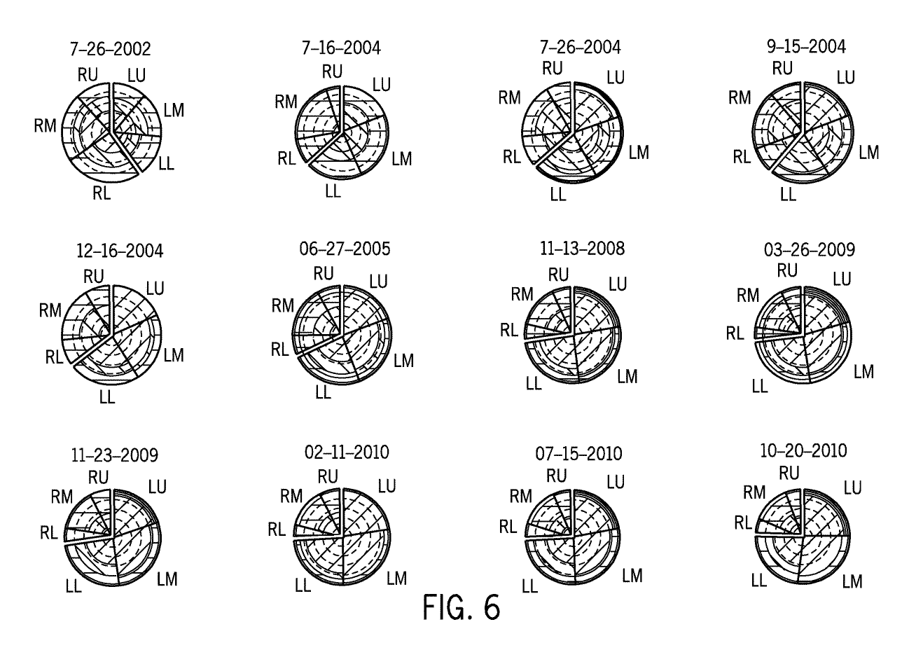

Referring now to FIG. 6, a glyph scheme is illustrated where a montage of glyphs are presented each with all the aforementioned characteristics such that each glyph represents the state in lung during an known instance of time, therapy, and or disease progression. More particularly, FIG. 6 shows the glyphs corresponding to a single patient's scan acquired at different time points.

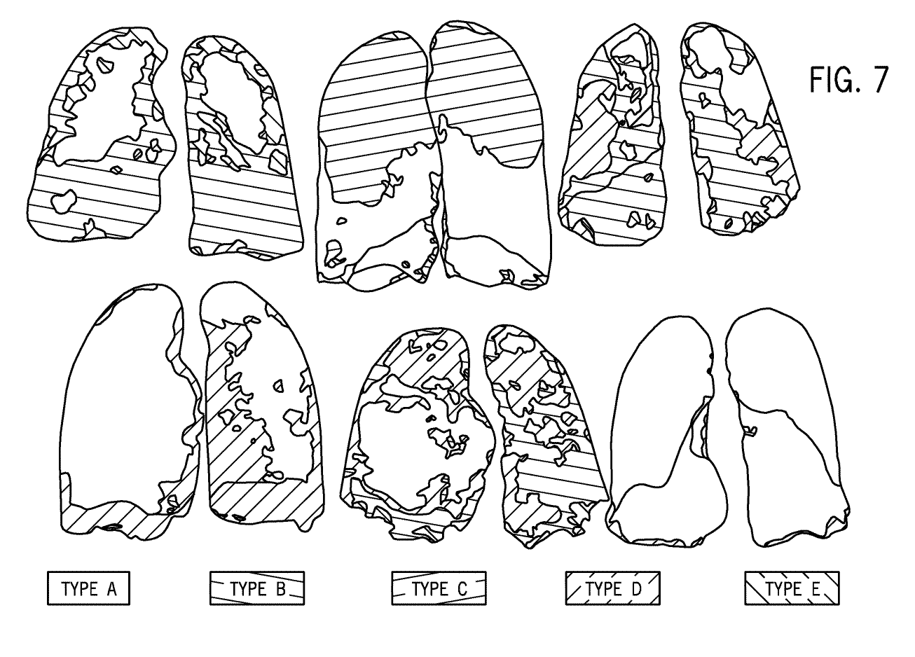

Referring to FIG. 7, a glyph scheme is illustrated where the coded disease states are displayed in a view/orientation dependent manner such that the tissue type that has maximum occurrence through the volume along that view is displayed. Such a presentation provides an unambiguous access point for optimal biopsy sites to harvest pathology tissue specimens. FIG. 7 shows the maximum disease projection for a number of independent patient-specific datasets.

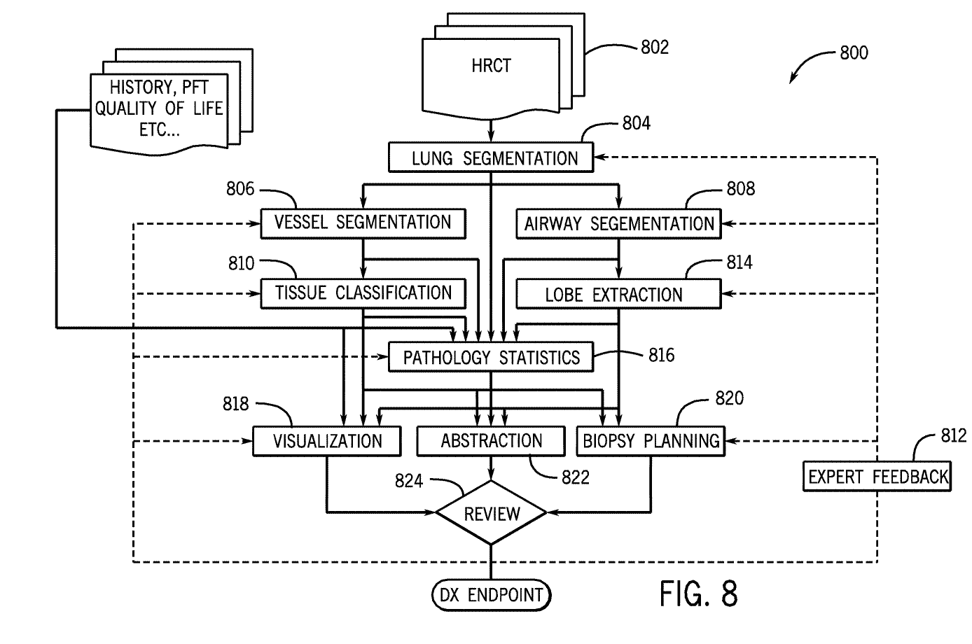

To achieve these and other results, a variety of techniques are employed. Referring now to FIG. 8, a flow chart illustrating processes of algorithmic subsystems of CALIPER is illustrated. As is evident from FIG. 8, the subsystems 800 behind CALIPER are quite complex. For example, High Resolution CT (HRCT) 802 or other medical imaging data may serve as a primary input that is provided to a plurality of segmentation components 804, 806, 808 for delineating lungs, vessels and airways, respectively. CALIPER advantageously includes a suite of algorithms to perform these tasks. In particular, CALIPER advantageously provides algorithmic integration via a cascade of dependency-resolved tasks, such that all segmentations can be performed concurrently. Compared to previous methods, this optimization reduces the computation time significantly. Mathematical morphology methods are used for this interleaved process. Accordingly, computational times on the order of only 1-2 minutes, as opposed to an hour by previous methods, are achieved.

Continuing with respect to FIG. 8, tissue classification, as indicated by process block 810 is performed. Given the visual acuity of the primal morphological disease-specific forms present in medical imaging data, lung tissue classification is typically cast into one of texture analysis, computer vision-based image understanding and content based information retrieval. Central to all these schemes was the selection of a representative expert labeled VOI of features, and providing this input to a classifier that is subsequently trained to (re)produce the expert labels. Descriptors based on histogram statistics, co-occurrence matrices, run length parameters, and fractal measures were typically used to enumerate the features. Artificial neural networks, Bayesian classifiers, and k-neighbor classifiers could also be used to classify the features.

To identify the similarity metric that best characterizes the expert grouping, a Multi Dimensional Scaling (MDS) may be used to project pairwise similarities between each of the VOIs. The multivariate similarity measure is projected into three dimensions, to visualize trends and groupings. Using the pairwise similarity matrix, MDS positions the data such that the Euclidean distances (other distances are also possible) between all pairs of the points in this plot reflect the observed distances as faithfully as possible. Parametric and non-parametric similarity metrics supported in “Volumetrics”, a plug-in module in the Analyze software, commercial available from the Mayo Clinic in Rochester, Minn., can be used. Parametric metrics included first and second order statistics and measures of effectiveness such as Fechner-Weber contrast measure, target-reference inference ratio, Fisher distance, and the like. Non-parametric similarity metrics were based on histogram distances such as Manhattan, Euclidean, Bhattacharya, Kolmogrov-Smirnoff and Cramer Von Mises (CVM) distance. Of all the metrics, MDS representation of CVM (the squared L2-metric between cumulative density functions) is advantageously consistent with expert groupings, such as illustrated in reference source 812 in FIG. 8.

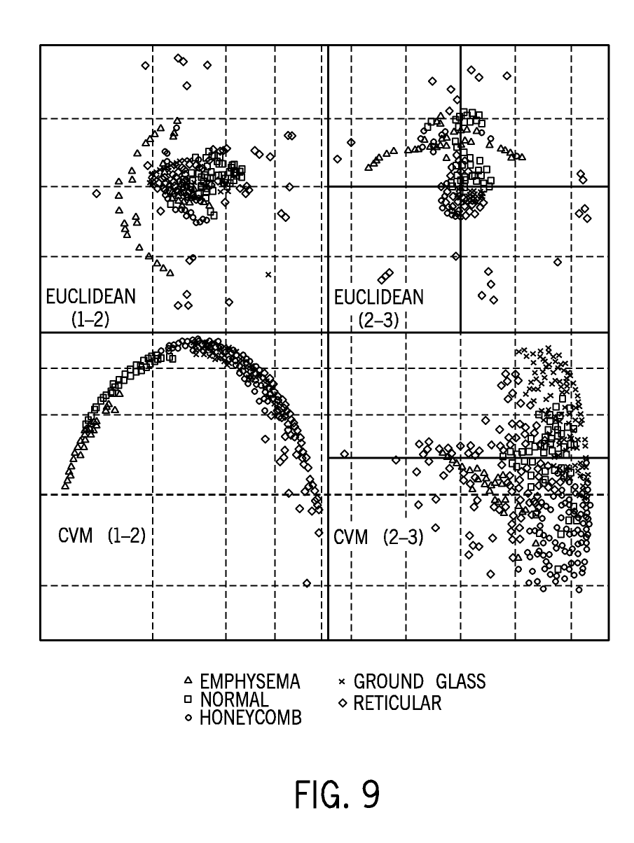

For example, FIG. 9 shows the axis1-axis2 (1-2) and 2-3 MDS projections for Euclidean and CVM similarity metrics, revealing the natural orderliness with which the VOIs, compared using Cramer Von Mises distance, aligns with the expert consensus. The honeycomb and ground glass features overlapping in the 1-2 projection are sufficiently separated in the 2-3 projection. As such, the use of CVM distance as a similarity metric to differentiate textures in image processing is particularly advantageous over previous methods.

Having established, albeit visually via MDS, that CVM distance could produce groupings statistically equivalent to expert consensus, the next step is to automatically cluster the CVM distance similarities and, hence, the VOIs into natural clusters, and then establish equivalence quantitatively. Previous clustering techniques (k-means, neural networks etc) typically needed explicit specification of the expected number of clusters. To create an unbiased stratification of VOIs, an unsupervised technique that automatically finds the natural number of clusters is preferred. Affinity propagation readily meets this stringent requirement. Briefly, affinity propagation uses message passing to iteratively find clusters given pair-wise similarities of n-dimensional data. In addition to resolving the clusters, it identifies the exemplar that is most ‘central’ to each of the clusters. In contrast to previous methods, affinity propagation is advantageously used herein to cluster intensity based appearance models. Clustering based on affinity propagation yielded five natural clusters and the groupings were highly correlated to the consensus groupings of experts as shown in the confusion matrix in Table 1.

TABLE 1

| | E | GG | HC | N | R |
|---|---|---|---|---|---|
| Emphysema (E) | 77 | 0 | 0 | 3 | 0 |
| GroundGlass(GG) | 0 | 137 | 1 | 2 | 10 |
| Honeycombing (HC) | 1 | 11 | 148 | 7 | 20 |
| Normal (N) | 0 | 0 | 0 | 265 | 0 |
| Reticular (R) | 0 | 16 | 32 | 0 | 246 |

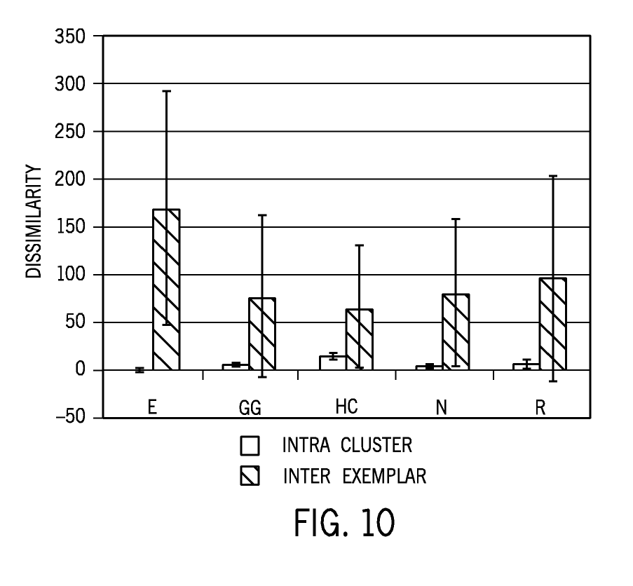

Referring to FIG. 10, the mean intra cluster and inter exemplar CVM distance values for this clustering are illustrated. Both results illustrate that affinity propagation based clustering of CVM similarity matrix yields a grouping consistent with expert consensus.

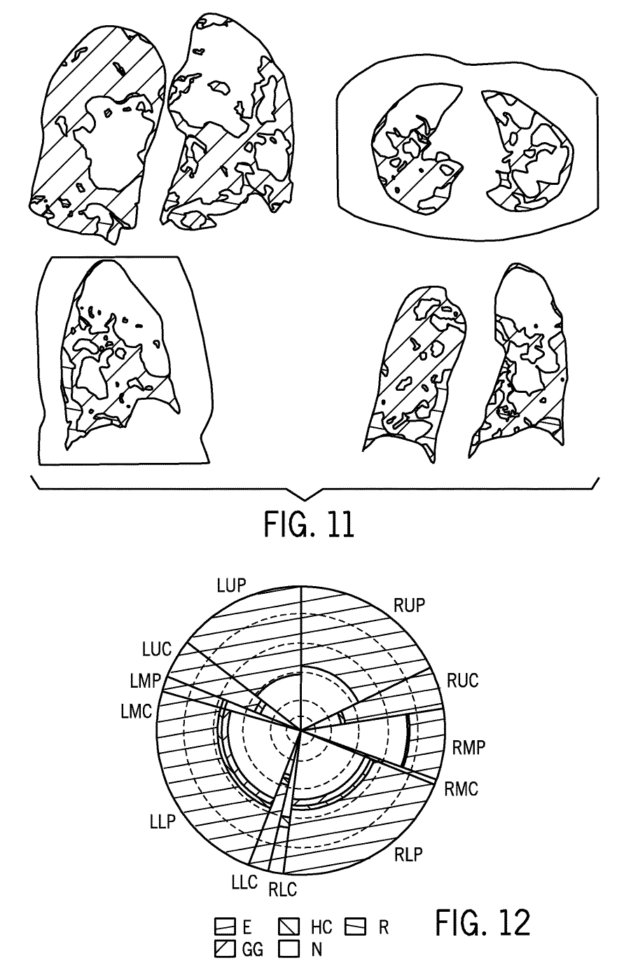

Referring again to FIG. 8 and, in particular, the tissue classification performed at process block 810, a local histogram in the neighborhood of the each lung voxel is compared with the exemplar and the key candidates at the borderlands between the classes using CVM similarity metric, and the label of the exemplar/borderland candidate that yields the minimum CVM is assigned to the voxel under examination. This approach has been applied to 730 datasets in the LTRC repository. Processing of all the datasets required approximately 25 hours; processing a single dataset required approximately two minutes. To process the same batch at 55 hours per dataset, the previous methods would have required 39,600 hours (1650 days; 4.5 years). As an example, FIG. 11 shows the classification results for a representative dataset. Visually, these results correlated with the EMD based algorithm currently undergoing validation by the LTRC community.

Given that the expert consensus can be emulated automatically using affinity propagation and CVM similarity, it is possible to automatically select the key VOIs across a more representative set of datasets. This can be accomplished with a VOI selection based on maximum dependency, maximum relevance, and minimum redundancy criterion. This can be used to assist in customizing the key VOIs across sites, scanners, acquisition protocols and reconstruction parameters.

It is noted that CVM has been used herein as an exemplary parameter; however, other parameters, including CVM-like metrics may be used to avail the system of differing strengths, weaknesses, opportunities, and failure modes of each of these metrics and their classifications thereof. As an alternate approach to reliably mimic the expert consensus, it is also possible to use a co-optative set of similarity metrics to favorably augment the efficacy of the above classification. In this approach, multiple pair-wise probability density function-based similarity metrics have been used. VOIs can be automatically grouped into natural clusters and relevant metrics were pruned based on the cluster's faithfulness to the disease-differentiating primal forms. The clusters from each of the relevant metrics may be independently refined for intra-partition compactness. The refined clusters may be aggregated into a super cluster using a cluster ensemble technique. The super clusters are validated against the expert consensus using Dice Similarity Metric (DSC). Using such comparisons, strong correlation of aggregations with those of experts has been shown. Also, a classifier based on such aggregated features can be used. In summary, by exploring the limits of creative tension, the lung classification algorithms in CALIPER bridges the gap between current computing constraints and the need for fast, robust, repeatable, and consistent tissue to disease characterization.

Referring again to FIG. 8 and, in particular, the lobe extraction performed at process block 814, the lobar extent of diffuse lung disease may be considered a highly-useful factor in the decision regarding lobar resection. However, automatic lobe extraction can still be a challenging problem, especially in the presence of incomplete fissures and pathology. To overcome these challenges, a probabilistic atlas of lobes is used based on an unbiased, reference-less shape stratification of the lungs similar to those used for grouping the left ventricles, referenced above and incorporated herein by reference. The lobes manually delineated by experts as part of the LTRC effort are embedded in this stratified space to create the probabilistic atlas. Physioanatomic based alignment of a specific lung onto this atlas provides reliable estimates of the lobes which can be further refined by incorporating the appearance model of the specific lung.

Continuing with respect to FIG. 8, the above-described analysis yields pathology statistics, as represented by process block 816, that are computed from the tissue classification across the different lobes and can be displayed in a number of ways. To be clinically useful for most situations, it is advantageous for this statistical information to be visualized, as represented by process block 818. While bar charts could be used to show the percentage distribution of the morphological patterns in the different lobes of the lungs, the layout of the information is not consistent with anatomic position, and they do not take into account the varying volumes of the individual lobes and whole lungs. Accordingly, the above-described glyph-based display techniques may be used. For example, FIG. 12 shows a representative glyph for an emphysematous lung. The glyph is divided into eleven circular sectors each representing one of the lobes; one lobe including relatively little data does not have a corresponding circular sector as described below. The lobes are uniquely labeled with three letters indicative of the three orthogonal directions. First letter (R/L) denotes respectively the right and left. The second letter (U/M/L) denotes respectively upper, middle and lower. The last letter (P/C) indicates respectively peripheral and central. The origin of the glyph is fixed at 12-o-clock starting with RUP lobe followed clockwise successively by RUC, RMP, RMC, RLP, RLC, LLC, LLP, LMC (which includes relatively little data and does not having a corresponding circular sector as describe above), LMP, LUC, and LUP lobes. Although no pleural separation demarcates the lingula from the remainder of the left upper lobe, this anatomic region is defined for the LTRC datasets. The asymmetry between the left and right lungs can be readily found in the glyph. The individual circular sectors span angles, or have areas, proportional to their respective lobe volumes. Within each circular sector, the distribution of diseases is represented by the color coded and radially offset arcuate segments, and the thickness or area of each segment is proportional to the corresponding disease's volume percentage presence in the corresponding lobe. The concentric circles are drawn at 20 percent intervals. For example, the left lower peripheral (LLP) lobe is 40 percent emphysematous, ˜55 percent normal and the remaining 5 percent is shared between ground glass and honey combing patterns. The radius of the big circle could be scaled proportionately to the total lung volume. Thus, within a single glyph, both global (total lung volume) and regional (lobe volume) functional capacity of the lung could be displayed concomitantly with the percentages of the patterns in the individual lobes.

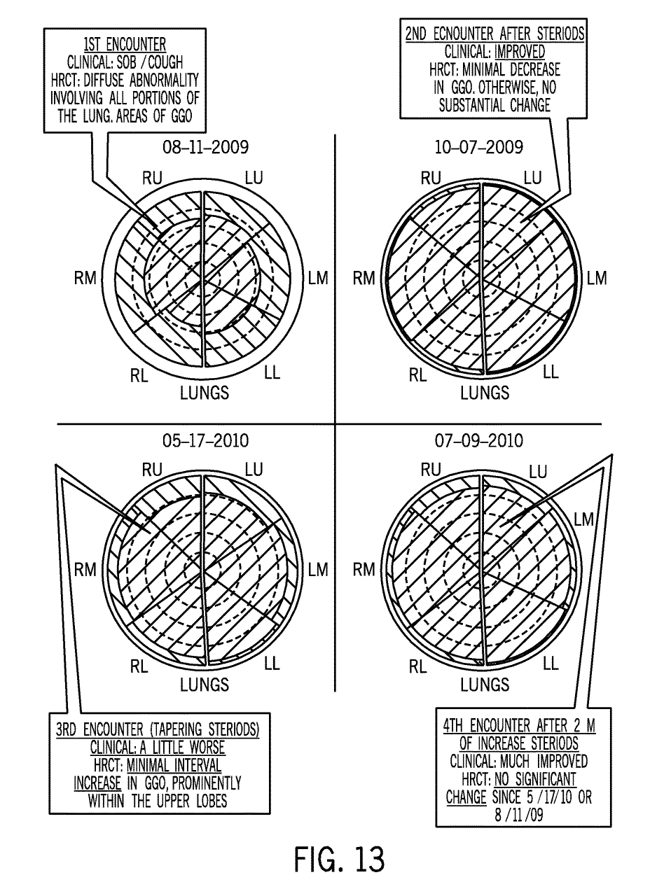

Referring to FIG. 13 and again FIG. 3B, the information can be displayed as a mosaic of glyphs from different CT scans highlighting the ease with which the intra patient disease distribution, or inter-patient disease distribution as a response to therapy, can be succinctly displayed. Additionally, the ethnicity, gender, age and height information of the patient can be used to find the normal values of functional parameters like FEV₁, FEV₆, FVC, PEF, FEF₂₅₋₇₅ using predicted normal equations. By inscribing or circumscribing the glyphs with a circle corresponding to normative lung volumes, a physician could instantly calibrate the subject's functional capacity in relation to the normal distributions. By making the glyphs iconic in that the different sectors and pie slices are hyperlinked to the corresponding raw data and its abstractions, CALIPER can provide a seamless level-of-detail navigation through the macro and micro characteristics of the lung, or other tissue volumes. Such a process may help multispecialty physicians make more accurate decisions on the status of patient's lungs. With robust, expeditious, reproducible characterization of the lung, lobes, airways, vessels and parenchymal tissues, accompanied by results summarized holistically as gleaned from both CT scans and from functional tests and presented in a consistent manner through a CALIPER like framework, the field of computer aided diagnosis may be advanced and elevated to a degree of maturity and universal applicability heretofore not evident.

This visualization can aid in a variety of clinical settings. One ready example is biopsy planning, such as represented by process block 820. When a histospecific classification of IPF is required, surgical lung biopsy is needed. HRCT scans and their quantitative characterization will help determine the optimal site for obtaining clinically and pathologically relevant tissue. For example, an ATS/ERS statement says “ . . . if the lung shows severe fibrosis with honeycombing the biopsy specimen should not be taken from the worst-looking areas . . . . However, if the lung does not show severe fibrosis or honeycombing grossly, the surgeon should take the biopsy from the abnormal areas of the lung”. The above-described processes and, in particular, glyph visualizations supports decision making for identifying the target lobe for biopsy. Furthermore, at the micro voxel level the regions of active concentration of abnormalities could be easily extracted and highlighted as adjuvant guides to the pathologist.

As another example, referring to the process of abstractions, as represented in FIG. 8 by process block 822, Tables 2a and 2b show the radiologic features associated with the differential diagnosis of idiopathic interstitial pneumonias.

TABLE 2a

| Clinical | Histologic | Usual Radiographic | Typical Distribution |
|---|---|---|---|
| Diagnosis | Pattern | Features | on CT |
| IPF/CFA | UIP | Basal-predominant reticular | Peripheral, |
| | | abnormality with volume | subpleural, basal |
| | | loss | |
| NSIP, | NSIP | Ground glass and reticular | Peripheral, |
| provisional | | opacity | subpleural, basal, |
| | | | symmetric |
| COP | OP | Patchy bilateral | Subpleural/ |
| | | consolidation | peribronchial |
| AIP | DAD | Progressive diffuse ground | Diffuse |
| | | glass density/consolidation | |
| DIP | DIP | Ground glass opacity | Lower zone, |
| | | | peripheral |
| | | | predominance in |
| | | | most |
| RB-ILD | RB | Bronchial wall thickening; | Diffuse |
| | | ground glass opacity | |
| LIP | LIP | Reticular opacities, nodules | Diffuse |

TABLE 2b

| Clinical | | |
|---|---|---|
| Diagnosis | Typical CT Findings | CT Differential Diagnosis |
| IPF/CFA | Reticular, honeycombing | Asbestosis |
| | Traction bronchiectasis/ | Collagen vascular disease |
| | bronchiolectasis; architectural | Hypersensitivity pneumonitis |
| | distortion. Focal ground glass | Sarcoidosis |
| NSIP, | Ground glass attenuation | UIP, DIP, COP |
| provisional | Irregular lines | Hypersensitivity pneumonitis |
| | Consolidation | |
| COP | Patchy consolidation and/or | Infection, vasculitis, |
| | nodules | sarcoidosis, alveolar |
| | | carcinoma, lymphoma, |
| | | eosinophilic pneumonia, |
| | | NSIP |
| AIP | Consolidation and ground | Hydrostatic edema |
| | glass opacity, often with | Pneumonia |
| | lobular sparing. | Acute eosinophilic |
| | Traction bronchiectasis later | pneumonia |
| DIP | Ground glass attenuation | RB-ILD |
| | Reticular lines | Hypersensivity pneumonitis |
| | | Sarcoidosis, PCP |
| RB-ILD | Bronchial wall thickening | DIP |
| | Centrilobular nodules | NSIP |
| | Patchy ground glass opacity | Hypersensitivity pneumonitis |
| LIP | Centrilobular nodules, ground | Sarcoidosis, lymphangitic |
| | glass attenuation, septal and | carcinoma, Langerhans' cell |
| | bronchovascular thickening, | histiocytosis |
| | thin-walled cysts | |

In Tables 2a and 2b, the following acronyms are used: acute interstitial pneumonia (AIP), cryptogenic fibrosing alveolitis (CFA); cryptogenic OP (COP); diffuse alveolar damage (DAD); desquamative interstitial pneumonia (DIP); idiopathic pulmonary fibrosis (IPF); lymphoid interstitial pneumonia (LIP); nonspecific interstitial pneumonia (NSIP); Pneumocystis carinii pneumonia (PCP); respiratory bronchiolitis-associated interstitial lung disease (RB-ILD); usual interstitial pneumonia (UIP).

Similar patterns of disease on HRCT of the lungs are available in other standards and review literature. CALIPER is able to take all the analyses and correlate them with the appropriate disease and provide all the results with a “proverai no droverai [trust but verify]” intent so that the physician has the complete information to make accurate decisions on a patient's wellness or disease.

Currently, given the computing uncertainties, the CT findings to disease mapping is qualitative. With the advent of CALIPER, the decision rules can be standardized through a quantitative measure. Repositories like the LTRC can be used in conjunction with CALIPER to explore empirical correlates between the current clinical practice and the holistic information provided by the glyphs. Such an exercise elucidates the clinical benefits of quantitative imaging and analysis of HRCT in the understanding and clinical management of DPLD.

As referenced above, the expert feedback 812 is incorporated throughout the above-described implementation. To this end, one goal of CALIPER is to serve as an imaging biomarker by phenotyping patients accurately, by establishing and managing disease more definitively, and by predicting prognoses. Given the effects of inter-subject variations, choice of data acquisition and reconstruction strategies, and lack of quantitative association of image-based decisions with clinical end points, it is important to incorporate analytic and clinical validation tools at the component level so that the strength, weakness and failure modes of each of the components can be precisely quantified and reported to the physician or the end user in the form of a measure of system confidence in the outcome.

Taken to a higher level of abstraction, this means that the components of an implementation of the CALIPER system advantageously possess (a) self-introspective mechanisms to assess their own performance, (b) the humanistic ability to learn, unlearn and relearn the decision rules under expert guidance or prior information, and (c) efficient processes to continually improve the performance with minimal burden to the practice. In general, CALIPER may include a review and feedback 824. CALIPER implementations support these crucial but heretofore neglected computational concepts, and this will accelerate the translation of this complex but realizable decision support system into routine clinical practice.

As described before, the appearance of a region around a lung voxel is enumerated by a feature and compared with the VOI exemplars/borderlands of a naturally clustered grouping. In this process, the feature space distance of the current voxel to the exemplar is computed. This distance can be statistically quantified using Mahalanobis distance to estimate the probability and hence confidence with which the tested voxel truly belongs to the same class as the exemplar/borderland. Aggregation of this statistic over the lung provides a confidence measure of the classification performed with respect to the reference VOIs selected. By ensuring that the training VOIs adequately cover the disease landscape, and by coupling the confidence measures with the glyphs, the analysis and summaries will have stronger correlation with the disease.

Through an interactive environment, the segmentation of the lung, vessels and airways could be edited and corrected by an expert. Longstanding experience with unlearning and relearning tools based on smart edits, smart edges, and shape propagation techniques has been leveraged to guide the segmentations towards perfection. In the case of the lobe extraction, the algorithm identifies the stratified lung space, learns the probabilistic locations of the lobes, and incorporates the appearance of the processed lung to refine, unlearn, and relearn the customizations required for the extraction of lobes in the specific lung CT scans.

The auto-learning ability of CVM based affinity propagation clustering of VOIs has already been described. The notion of unlearning and relearning in unsupervised classifiers through expert-in-the-loop guidance is also provided. Such techniques are extremely valuable to the ultimate acceptance of the results by experts. Towards this, CALIPER has the ability to cooperatively learn, train, classify and annotate the key signatures associated with the disease-specific patterns. This is done through a student-mentor paradigm wherein the (adaptively learning) computer/algorithm (student) identifies (based on peer review, guidelines, specification etc.), and groups disparate regions in a plurality of patient-specific scans, selects key signatures from the groups, assesses the efficacy of the grouping through a domain expert (mentor), refines the grouping and propagates the learning to the other datasets. Through preparation, the algorithm proactively engages the mentor to reinforce and refine its understanding. Additionally, by selectively clarifying the interrogation space, and ensuring adequate coverage of the same, the student engages the mentor in an effective way. The mentor in turn, motivated by the proactive inclination of the student, enthusiastically participates in the intellectual exchange.

Through judicious combination of pedagogical tools and efficient computer adapted testing (CAT) methodologies, the student-mentor paradigm described here overcomes the drawbacks of the previous supervisor-workhorse paradigm.

Additionally, it provides an intellectual and trustworthy workflow for automating and validating routine radiological readings. This timely breakthrough maximizes the strength of imaging, image analysis and domain expert interpretation paving the way for enhanced personalized, predictive, preemptive and participatory radiology. Though truly disruptive, the technology has strong self-attested predicates and integrates seamlessly with the clinical workflow.

The scenario described above is the process followed in spatiotemporal and population-independent computer adapted standardized scholastic assessment of individuals with reference to a peer group. These tests are administered based on variants of Item Response Theory (IRT)—a statistical framework based on the idea that the probability of getting an item (question) correct is a function of person and item parameters. Person parameters represent the student's ability to correctly answer the question. Item parameters include difficulty of the item, “guessability”, and discrimination. Using CAT, the ability of the examinee can be iteratively estimated which in turn can be used in the selection of subsequent queries. By such adaptive tailoring of the questions, maximal information about the examinee's ability levels can be elicited at reduced standard estimation errors and greater precision with a minimal set of key questions.

In the context of CALIPER, the computer/physician can be interchangeably treated as examiner/examinee. By changing the abstraction functions and the results thereof, multiple examinees can be obtained. The Mahalonobis distance between a given signature and its nearest exemplar gives the confidence and hence the difficulty of identifying the signature. Discriminability of a signature is a function of its distance to the borderlands across different clusters. The difficulty and discriminability can be pre computed and the complexity can be ascertained with the examiner. By investigating the concordance between the response of the examinee and the examiner, the efficacy of the algorithm/rater can be assessed. More importantly, because this a learning environment as opposed to conventional CAT environment, the examinee can relearn or unlearn the decision making, thereby further increasing the ultimate reliability of the automated diagnosis of medical images. The opportunities with such a closed-ended assessment system are essentially endless.

CALIPER can evaluate DPLD disorders that have variable radiographic appearances and clinical phenotypes. Both the radiographic evaluation and clinical characterization are difficult, and CALIPER is aimed at consistently quantifying and characterizing these abnormalities to prove that with expert physician feedback and a flexible and trainable algorithm, the clinical confidence in the diagnosis, consistency of the imaging evaluation, and quality of the reporting of disease can be improved. In turn, confidence in the algorithm and its output can be leveraged for novice physician training and more consistent use of descriptive terms for the characterization of disease. With the philosophy of keeping the expert physician “in the loop” and improving the quality of the algorithm output, the highly trained algorithm then becomes a physician-trainer.

The majority of previous expert systems and associated quantitative tools depend on strictly controlled image acquisition protocols to provide consistent results. For example, even simple quantification of easily visually recognizable and grossly apparent diffuse abnormalities, such as pulmonary emphysema, through a process of pixel counting is extremely sensitive to slice thickness, acquisition parameters, and the reconstruction kernel utilized. It has been shown that the detected quantity of these abnormalities can be affected more than 50 percent depending on the acquisition and reconstruction parameters. Through careful training and robust algorithmic design, CALIPER could be less affected by reconstruction and scan parameters. With incorporation of noise immunity, CALIPER could facilitate useful processing of images obtained at lower administered dose. Processing low dose CT datasets could be of great benefit to future research studies, since currently the highest allowable dose is often utilized to assure consistent high quality imaging for the purposes of reproducible quantitative analysis, even though this may be more necessary for visual clinical diagnosis.

CALIPER embodies a few specific foundational principles and features, such as providing a seamless integration of multidimensional and multispecialty data. Multispecialty data includes patient history (age, sex, ethnicity etc), physical examination (height, weight, and the like), and clinical-application-specific information, such as pulmonary function tests, chest radiology scans, and where available, pathology data and reports. CALIPER also embodies an aggregated analysis of multispecialty data. The critical information present in and derived from the multispecialty data is aggregated as per clinical guidelines and established clinical pathways to provide a comprehensive, high level view of a patient and, specifically, the region of interest, such as the lung. CALIPER further embodies a robust and fast, high-resolution based tissue quantification mechanism. This includes algorithms for tissue volume, including whole lung, airway and vessels, lobe segmentation, lung tissue classification and associated statistics. Classification emulates multi-radiologist consensus by judiciously aggregating the clusters from multiple feature descriptors. CALIPER also embodies optimal site specification for surgical biopsy. In situations where a definitive diagnosis of, for example, DPLD, is required, the tissue classification can be used to determine the optimal site(s) for biopsy. CALIPER additionally embodies an executive, iconic level-of-detail summary of tissue wellness. The power of advanced visualization methods is exploited to provide a macro-to-micro view of tissue pathology. At the macro level, the structural and functional information is summarized into a “glyph” that can be readily interpreted and correlated to known disease states. At the micro level, the tissue scans is overlaid with color coded classification and confidence measures. Further still, CALIPER provides a clinically expedient summary. Clinical expedience refers to the accuracy, precision, and speed with which the summary report is generated. A highly accurate and precise tissue quantification is achieved within, for example, a minute using a standard modern computer workstation, such as described above.

In addition, CALIPER provides a verifiable summary. At least three levels of verification are featured in CALIPER. At the micro level, the classification algorithm associates a confidence measure to each of the classified voxels. At the macro level, the different regions of the iconic summary are linked to the underlying data and abstractions to help the physician navigate through and confirm the findings. At the system level, the overall performance of CALIPER can be assessed using a facile physician-in-the-loop paradigm based on the principles of standardized computer adapted tests, with future results modified by the physician in the loop feedback.

Finally, CALIPER is designed to reliably work across an acceptable range of clinically valid imaging modalities, reconstruction protocols, and general image and manufacturer types, including those produced by multiple different vendors and brands of imaging systems.

As described above, beyond the capabilities of previous CAD-type and other systems, CALIPER is designed to seamlessly embed proof-of-efficacy analytical and clinical validation tools to facilitate the accelerated translation of CALIPER into routine clinical practice and validate the utility of CALIPER for improved patient care. As will be detailed, CALIPER is capable of operating as an intelligent router of patient specific datasets to the most appropriate radiology specialists in a night-hawking teleradiology environment where, currently, the images are served to the physicians on a first come first reviewed basis irrespective of the physician's exposure to the patient-specific cues. CALIPER is also capable of operating as a holistic environment that helps build a quantitative automatic consensus on the patient's tissue volume state, such as lung state, as gleaned from multidisciplinary data and a diagnostic and prognostic tool that helps to track the course of treatment. Further still, CALIPER facilitates the realization of these positions to optimize the medicine at large. CALIPER replicates the humanistic trait, skill, courage, and optimism to embrace good ideas (algorithms/metrics/training sets) and not remain imprisoned by bad ones.

The present invention has been described in terms of one or more preferred embodiments, and it should be appreciated that many equivalents, alternatives, variations, and modifications, aside from those expressly stated, are possible and within the scope of the invention.

We claim:

1. A system comprising:
a medical imaging device generating a plurality of tissue data including a plurality of patient-specific multi-dimensional volumetric representations of a tissue volume of at least one region of interest (ROI) within at least one in vivo tissue of a patient;
a server hardware computing device coupled to the medical imaging device via a network and comprising at least one processor executing program instructions within a memory, which, when executed, cause the system to:
decode, from the plurality of tissue data, a plurality of data types, wherein the program instructions analyze each of the plurality of data types via a cascade of dependency-resolved tasks;
perform a segmentation process to identify a plurality of tissue types, each associated with one of the plurality of data types;
classify, using a similarity metric analysis identifying an affinity of each of the plurality of tissue types with at least one previously identified tissue type, each of the plurality of tissue types as a normal or abnormal tissue type;
automatically generate a plot including a plurality of tissue type clusters for the regional and temporal distribution of each of the normal and abnormal tissue types in the ROI; and
automatically generate a graphical user interface (GUI) displaying, on a hardware display device coupled to the network, a report comprising a plurality of shapes, the area of each of the plurality of shapes being proportional to the amount and distributions of data in each of the plurality of tissue type clusters.

2. The system of claim 1, wherein the similarity metric includes a multi-dimensional scaling representation of Cramer Von Mises distance between a plurality of points of the plurality of tissue data.

3. The system of claim 1, wherein the plurality of shapes:
represent the plurality of tissue data;
comprise at least one circular glyph further comprising a plurality of arcuate segments, each of which includes an area proportional to an amount of data in each of the plurality of tissue type clusters.

4. The system of claim 1, wherein the report displays the plurality of tissue data as a circular-shaped glyph comprising a plurality of circular sectors, each including:
an overall area proportional to the tissue volume of a corresponding ROI; and
a plurality of radially offset arcuate segments, which:
together define the overall area of a circular sector; and
each include an area proportional to an amount of data in each of the plurality of tissue type clusters within the corresponding ROI.

5. The system of claim 1, wherein the at least one shape corresponds to at least one anatomic features represented by the tissue data.

6. The system of claim 5, wherein a first shape of the plurality of shapes is positioned concentrically with a second shape of the plurality of shapes wherein:
the first shape represents a distribution of normal tissue of the anatomic features; and
the second shape represents a distribution of abnormal tissue of the anatomic features.

7. The system of claim 5, wherein the plurality of shapes together define an area representative of anatomic functionality compared to a normal anatomic functionality within a population.

8. The system of claim 1, wherein the program instructions further cause the system to display on the GUI, within the report, a history of disease progression within the patient.

9. The system of claim 1, wherein generation of the plot of the plurality of tissue type clusters includes affinity propagation.

10. The system of claim 1, wherein the report includes a plurality of patient demographic information representing a predicted tissue volume of the patient.

11. The system of claim 1, wherein the report includes a display of:
a maximum disease projection; and
a data type having a maximum occurrence in the plurality of tissue data.

12. A system comprising:
a medical imaging device generating a plurality of tissue data including a plurality of patient-specific multi-dimensional volumetric representations of a tissue volume of at least one region of interest (ROI) within at least one in vivo tissue of a patient;
a server hardware computing device coupled to the medical imaging device via a network, and comprising at least one processor executing instructions within a memory, which, when executed, cause the system to:
decode, from the plurality of tissue data, a plurality of data types, wherein the program instructions analyze each of the plurality of data types via a cascade of dependency-resolved tasks;
perform a segmentation process to identify a plurality of tissue types, each associated with one of the plurality of data types;
classify, using a similarity metric analysis identifying an affinity of each of the plurality of tissue types with at least one previously identified tissue type, each of the plurality of tissue types as a normal or abnormal tissue type;
automatically generate a plot including a plurality of tissue type clusters for the regional and temporal distribution of each of the normal and abnormal tissue types in the ROI; and
automatically generate a graphical user interface (GUI) displaying, on a hardware display device coupled to the network, a report including a circular-shaped glyph including a plurality of circular sectors, each including:
an overall area proportional to the tissue volume of a corresponding ROI; and
a plurality of radially offset arcuate segments, which:
together define the overall area of a circular sector; and
each include an area proportional to an amount of data in each of the tissue type clusters within the corresponding ROI.

13. The system of claim 12, wherein:
the at least one ROI includes:
a first spatial portion having a first volume; and
a second spatial portion having a second volume; and
the plurality of radially offset arcuate segments include:
an inner arcuate segment having a first area proportional to the first volume; and
an outer arcuate segment having a second area proportional to the second volume.

14. The system of claim 12, wherein the plurality of tissue types include a healthy tissue and a diseased tissue.

15. The system of claim 12, wherein:
the at least one ROI includes:
a first ROI having a first volume;
a second ROI having a second volume and neighboring the first ROI;
the plurality of circular sectors include:
a first circular sector having a first overall area proportional to the first volume; and
a second circular sector having a second overall area proportional to the second volume, and the second circular sector neighboring the first circular sector.

16. The system of claim 12, wherein the circular sectors include a plurality of colors representative of spatial and quantification information of the plurality of tissue data.
